(19) United States
(12) Patent Application Publication
(10) Pub. No.: US 2024/0216273 A1
(43) Pub. Date: Jul. 4, 2024

(54) TOPICAL ANESTHETIC

(71) Applicant: Primera Compounding, LLC, Rosenberg, TX (US)

(72) Inventors: Paula Hooper, Rosenberg, TX (US); Paramita Sarkar, Rosenberg, TX (US); Basil Raad, Rosenberg, TX (US)

(21) Appl. No.: 18/608,602

(22) Filed: Mar. 18, 2024

Related U.S. Application Data
(63) Continuation-in-part of application No. 18/345,674, filed on Jun. 30, 2023.
(60) Provisional application No. 63/491,161, filed on Mar. 20, 2023.
(60) Provisional application No. 63/358,410, filed on Jul. 5, 2022.

(51) Int. Cl. A61K 9/107; A61K 9/00; A61K 31/167; A61K 31/245; A61K 47/18; A61K 47/24; A61K 47/26; A61K 47/38

(52) U.S. Cl. CPC A61K 9/1075; A61K 9/0014; A61K 31/167; A61K 31/245; A61K 47/186; A61K 47/24; A61K 47/26; A61K 47/38

(57) ABSTRACT

Compositions for a viscous topical anesthetic for use in in-office treatments or surgical procedures are described. The compositions comprise a mixture of two anesthetic agents incorporated into a positively charged nanoparticle and suspended in a viscosifying solution. The described compositions increase the dispersion and absorption of the anesthetics.

### CROSS REFERENCE TO RELATED APPLICATIONS

This application claims priority to U.S. Provisional Patent Application Ser. No. 63/491,161 filed on Mar. 20, 2023. This application is a continuation-in-part of U.S. patent application Ser. No. 18/345,674 filed on Jun. 30, 2023, which claims priority to U.S. Provisional Patent Application Ser. No. 63/358,410 filed on Jul. 5, 2022. The entire disclosure of that patent application is part of the disclosure of the present application and is hereby incorporated by reference in its entirety.

### FIELD OF THE INVENTION

The present invention relates generally to an ophthalmic anesthetic. More specifically, the invention relates to a topical anesthetic having a mixture of two anesthetic agents incorporated into positively charged nanoparticles.

### BACKGROUND OF THE INVENTION

Topically applied anesthetic agents are typically used to prevent the transmission of nerve impulses without causing unconsciousness. They generally act by reversibly binding to fast sodium channels from within nerve fibers to prevent sodium from entering the nerve fibers. Typically local anesthetics have the suffix “-caine” in their names and are usually either ester- or amide-based. Ester-based local anesthetics (such as procaine, amethocaine, cocaine, benzocaine, and tetracaine) are generally fast-acting, but are rapidly hydrolyzed by plasma and tissue esterases. Amide-based local anesthetics (such as lidocaine, mepivacaine, etidocaine, ropivacaine, and bupivacaine) are degraded in the liver and are typically longer lasting.

Lidocaine and tetracaine are used a local anesthetic in various ophthalmic procedures (e.g., cataract surgeries, corneal abrasions, refractive surgeries, or retinal injections), minor dermal procedures (e.g., skin biopsies or suturing), and intranasal procedures.

There is a continuing need for the development of safe topical anesthetics to improve patient comfort and safety in various surgeries and procedures.

### SUMMARY OF THE INVENTION

The present invention relates generally to compositions for topical anesthetic agents. More specifically, the invention relates to topical anesthetic compositions for use in in-office treatments or surgical procedures.

According to an embodiment, a topical anesthetic composition comprises: a mixture of at least two anesthetic agents, wherein one anesthetic agent is an ester-based anesthetic agent and the other anesthetic agent is an amide-based anesthetic agent; a plurality of lipid-based positively charged nanoparticles; and a viscous base.

The ester-based anesthetic agent can be selected from the group consisting of procaine, amethocaine, benzocaine, or tetracaine. In one embodiment, the ester-based anesthetic agent can be tetracaine HCl. The tetracaine HCl can be present in concentrations ranging from about 0.01% w/w to 4.0% w/w.

The amide-based anesthetic agent can be selected from the group consisting of lidocaine, mepivacaine, etidocaine, ropivacaine, or bupivacaine. In one embodiment, the amide-based anesthetic agent can be lidocaine HCl. The lidocaine HCl can be present in concentrations ranging from about 0.05% w/w to 6.0% w/w.

The topical anesthetic composition further comprises a preservative. In one embodiment, the preservative can be benzalkonium chloride.

The viscous base comprises a cellulose base. The cellulose base can be selected from the group consisting of a hypromellose or hydroxypropyl methylcellulose, a microcrystalized cellulose, a methylcellulose, and mixtures thereof.

The viscous base further comprises a cation selected from the group consisting of cetylpyridinium chloride, cetyltrimethylammonium bromide, cetalkonium chloride, and mixtures thereof.

The viscous base further comprises one or more emulsifiers. In one embodiment, the emulsifier can be lecithin.

The viscous base further comprises a nonionic surfactant. In one embodiment, the nonionic surfactant can be polysorbate 80.

In one embodiment, the plurality of positively charged nanoparticles are liposomes. The liposomes can include a mixture of a lecithin, a polysorbate surfactant, and a cation selected from the group consisting of cetylpyridinium chloride, cetyltrimethylammonium bromide, cetalkonium chloride, and mixtures thereof.

In another embodiment, the plurality of positively charged nanoparticles are nanoemulsions. The nanoemulsions can include a mixture of mineral oil, a lecithin, a polysorbate surfactant, and a cation selected from the group consisting of cetylpyridinium chloride, cetyltrimethylammonium bromide, cetalkonium chloride, and mixtures thereof.

The topical anesthetic composition further comprises sterile water for injection and to obtain a desire weight for the composition. The pH of the topical anesthetic composition can be adjusted to about 4.5-6.5 using hydrochloric acid or sodium hydroxide.

### DETAILED DESCRIPTION OF ILLUSTRATIVE EMBODIMENTS

While the specification concludes with the claims particularly pointing and distinctly the invention, it is believed that the present invention will be better understood from the following description. The present invention can comprise the components of the present invention as well as other ingredients or elements described herein. As used herein, “comprising” means the elements recited, or their equivalent, plus any other element or elements which are not recited. The terms “having,” “including,” and “comprised of” are also to be construed as open ended unless the context suggests otherwise.

The term “about”, as used herein, refers to a +/−10% variation from the nominal value. It is to be understood that such a variation is always included in a given value provided herein, whether or not it is specifically referred to. All ranges recited herein include the endpoints, including those that recite a range “between” two values. Terms such as “about,” “around”, “generally,” “substantially,” and the like are to be construed as modifying a term or value such that it is not an absolute. Such terms will be defined by the circumstances and the terms that they modify as those terms are understood by those skilled in the art. This includes, at the very least, the degree of expected experimental error, technique error and instrument error for a given technique used to measure a value.

One or more embodiments of the invention relate to a viscous topical anesthetic composition for use in less complicated surgical procedures that do not require general anesthesia, such as, in-house or relatively quick surgical procedures. In one or more embodiments, the topical anesthetic composition comprises at least two anesthetic agents and lipid-based positively charged nanosized particles (or liposomes) or an emulsion/nanoemulsion (used interchangeably herein) incorporated into a viscous cellulosic base. The liposomes/emulsion further comprise a cation, a nonionic surfactant and an emulsifier. The topical anesthetic composition can include a preservative. Sterile water can be injected into the composition to reach a desired weight for the composition. The topical anesthetic composition can further optionally include hydrochloric acid or sodium hydroxide to adjust its pH to about 4.5-6.5. An exemplary suspension base is described in Applicant's Ser. No. 18/345,674, which is incorporated by reference herein.

The topical anesthetic composition can be used in a variety of dermatological applications and is suitable for applications at needle stick sites. The topical anesthetic composition is generally applicable in ophthalmological, nasal, and dermatological minor surgeries. In one exemplary embodiment, the topical anesthetic composition can be used in cataract surgery. The mixture of the anesthetic agents in a viscous base comprising liposomes ensures better wetting of the cornea and longer duration of analgesia. The one or more embodiments of the invention increase both dispersion and absorption of the active pharmaceutic ingredients on the surface of the eye to enhance the therapeutic anesthetic effects desired. The topical anesthetic composition of the present invention exhibits an unexpected increase in its effective time over known anesthetic compositions.

Specifically, the one or more embodiments of the present invention represent a combination of an ester-based anesthetic agent such as tetracaine HCl and an amide-based anesthetic agent such as lidocaine or lidocaine HCl to achieve an unexpected enhancement in the delivery and onset of anesthesia when incorporated into the viscous base comprising micro-fluidized positively charged liposomes.

Preferably one of the anesthetic agents is a fast acting ester-based local anesthetic agent while the other anesthetic agent is a longer acting amide-based anesthetic agent. In one or more embodiments the ester-based anesthetic agent can be selected from the group consisting of procaine, amethocaine or tetracaine; while the amide-based anesthetic agent can be selected from the group consisting of lidocaine, mepivacaine, etidocaine, ropivacaine, or bupivacaine.

One preferred embodiment of the ester-based local anesthetic agent in the topical anesthetic composition is tetracaine hydrochloride (HCl) at a concentration ranging from 0.01% w/w to 4.0% w/w of the composition. A preferred embodiment of the amide-based local anesthetic agent in the topical anesthetic is lidocaine HCl at a concentration ranging from 0.05% w/w to 6.0% w/w of the composition. One embodiment of the local anesthetic composition contains about 0.5% w/w tetracaine HCl and about 1.0% w/w lidocaine HCl. The topical anesthetic composition can exhibit an enhanced therapeutic effect due to the synergistic effect of the two anesthetic agents.

Preferably, the preservative is a cationic surfactant having antimicrobial properties. An exemplary preservative is benzalkonium chloride.

Viscosity is another important factor that can affect the efficacy and duration of both topical tetracaine and lidocaine. Viscosity refers to the thickness or resistance to flow of a liquid and can impact the ability of a topical anesthetic to penetrate the skin and remain in contact with the nerve endings for a prolonged period. Higher viscosity formulations of topical anesthetics have been shown to improve the efficacy and duration of anesthesia. This is because higher viscosity formulations tend to stay on the skin for longer periods of time, which can increase the depth and duration of the anesthetic effect. This can be particularly important for procedures that require prolonged anesthesia, such as surgical procedures or longer-lasting pain relief. In addition, viscosity can also impact the rate of drug release from the formulation. Higher viscosity formulations may release the drug more slowly, leading to a more sustained and prolonged anesthetic effect. This can be beneficial for reducing the need for repeated applications of the anesthetic.

However, it's worth noting that the optimal viscosity for a given formulation may depend on various factors, such as the specific mixture of anesthetics used, the intended use, and the properties of the skin being treated. In some cases, lower viscosity formulations may be more appropriate for achieving a faster onset of anesthesia or improving skin penetration. Therefore, different embodiments of the topical anesthetic composition of the present invention will be formulated to have different viscosities. In one or more embodiments, the topical anesthetic composition has a viscosity from about 100-800 cP.

The viscosity of the topical anesthetic composition can be adjusted by using one or more cellulose derived polymeric bases and/or manipulating the concentration of the cellulose derived base. In one exemplary embodiment, the base comprises hydroxypropyl methylcellulose (HPMC) used at 0.2% to 1.1% by weight of the nanoparticle (w/w). In other embodiments, the cellulose base can be microcrystalline cellulose, hydroxyethylcellulose, methylcellulose or mixtures thereof.

The anesthetic agents are carried by the nanoparticles to the cell surface through electrostatic forces and physical interactions. Accordingly, the beneficial properties of the nanoparticles (i.e., even dispersion and increased absorption) are adopted by the anesthetic agents.

Multiple embodiments of the topical anesthetic composition include positively charged lipid-based nanosized particles made from either liposomes or an emulsion incorporated into the cellulosic base. Liposomes are spherical nanoparticles that have an aqueous compartment enclosed by a bimolecular phospholipid membrane. Emulsions, on the other hand, are stable suspensions of small droplets of one liquid in another liquid with which it is immiscible and stabilized by an emulsifying agent as described above. The nanoparticles can be designed with a surface charge that helps dispersion in both solutions and suspensions. Additionally, the size of nanoparticles can be fine-tuned by manipulating the formation conditions. For example, micro-fluidization under certain conditions can generate liposomes of a uniform size.

Lipid-based nanoparticles can encapsulate or effectively adsorb both hydrophilic and hydrophobic anesthetic agents, such as lidocaine and tetracaine. The lipid-based nanoparticles facilitate their transport across the cell wall, leading to improved drug delivery and prolonged anesthetic effect. Such nanoparticles can protect the anesthetic agents from degradation and enhance their penetration into the cell, resulting in prolonged anesthetic effect.

One embodiment of a liposomal nanoparticle includes a mixture of: an emulsifier, such as, lecithin; a nonionic surfactant, such as, a polysorbate; and a cation selected from the group consisting of a cetylpyridinium halide, a cetrimonium halide, a cetalkonium halide, or mixtures thereof. An embodiment of the nanoemulsion can include a mixture of mineral oil, a lecithin, a polysorbate surfactant, and a cation selected from the group consisting of cetylpyridinium chloride, cetyltrimethylammonium bromide, cetalkonium chloride, and mixtures thereof.

Sterile water can be included with lecithin. HPMC has a concentration of about 0.2%-1.1% by weight of the total weight of the nanoparticle (w/w). The lecithin can be a complex mixture of soy derived phospholipids, glycolipids, and triglycerides having a concentration of about 0.1%-0.5% by weight of the total weight of the nanoparticle (w/w). The polysorbate can be polysorbate 80 having a concentration of about 0.1%-0.5% by weight of the total weight of the nanoparticle (w/w). The cetylpyridinium halide may be cetylpyridinium chloride having a concentration of about 0.002% by weight of the total weight of the nanoparticle (w/w). The cetrimonium halide may be cetrimonium bromide. The cetalkonium halide may be cetalkonium chloride.

Positive surface charges ensure that the nanoparticles are evenly dispersed through electrostatic repulsion. Certain embodiments use cetylpyridinium chloride (CPC) alone or with other suitable cationic molecules, such as cetrimonium bromide (CTAB) or cetalkonium chloride (CKC) to provide the nanoparticles with the appropriate positive charge. The size of the nanoparticles is typically homogenized by conventional micro-fluidization techniques to further promote even dispersion in the composition. Following micro-fluidization, the size of the nanoparticles will typically range from about 50 nm to about 200 nm in diameter.

Advantageously, the nanoparticles are carried by the base with variable viscosity to facilitate the nanoparticles lingering on the cell surface for longer, thereby increasing the chance for uptake.

A preferred embodiment of the topical anesthetic composition includes tetracaine HCl 0.5%, lidocaine HCl 1%, benzalkonium chloride 0.005%, sterile water for injection, a liposomal nanoparticle with cetylpyridinium chloride, lecithin, polysorbate 80, and a hydroxypropyl methylcellulose base. Optionally, the topical anesthetic composition can include hydrochloric acid or sodium hydroxide to adjust the pH to around 4.5-6.5. The composition can be brought to a final desired weight with sterile water.

In one embodiment, a kit comprises one or more pre-sterilized bottles or containers containing the topical anesthetic composition disclosed herein. For example, the kit can include three pre-sterilized single dose containers containing the topical anesthetic composition disclosed herein.

In an embodiment, the topical anesthetic composition can be made by: a) mixing two anesthetic agents with a preservative in sterile water to form a Phase I solution; b) preparing positively charged nanoparticles in a viscous base to form a Phase II dispersion; c) mixing the Phase I and Phase II solutions for about 5 minutes to 60 minutes; and d) processing the mixed phases through a microfluidizer. The pH of the resulting dispersion is adjusted to 4.5-6.5 using hydrochloric acid or sodium hydroxide and adjusting the final weight of the composition with injected sterile water.

The Phase I solution can include a mixture of the two anesthetic agents and a cationic surfactant in sterile water. The Phase I solution represents about 5-15% w/w of the final composition and is made by adding about 0.2% w/w to 2.0% w/w of the composition of an ester-based anesthetic agent and about 0.5% w/w to 4.0% w/w of an amide-based anesthetic agent to sterile water and continuously mixing the solution until the anesthetic agents are dissolved. A preservative at about 0.005% w/w to 0.01% w/w of the composition is then added to the solution while mixing.

One exemplary embodiment of the Phase I solution is made using: tetracaine HCl at a concentration ranging from 0.2% w/w to 2.0% w/w of the composition; lidocaine at about 0.005% w/w to 0.01% w/w of the composition; and benzalkonium chloride at a concentration ranging from 0.005% w/w to 0.01% w/w of the composition; and sterile water.

The positively charged nanoparticles of Phase II are typically lipid-based nanoparticles (liposomes) or an emulsion. The liposomes or emulsion can be incorporated into a cellulous derived base.

The liposomal nanoparticles are typically formed from a combination of a nonionic surfactant; a cation such as a cetylpyridium halide, a cetrimonium halide, a cetalkonium halide, or mixtures thereof; lecithin and sterile water for injection. Some embodiments of the lipid-based nanoparticles use a lecithin that is a complex mixture of soy derived phospholipids, glycolipids, and triglycerides. The cationic compound is chosen so that the solvent exposed surface area of the nanoparticle carries a positive charge. One embodiment of a liposome is made as follows: step (a) mix a surfactant such as polysorbate 80 (0.1% to 0.5% w/w) and a cation such as cetylpyridinium chloride (0.02% w/w); step (b) in a separate container mix lecithin (0.1% to 0.5% w/w) with sterile water; and step (c) mix the emulsifier and cation mixture from step (a) with the lecithin phase from step (b).

An embodiment of the Phase II dispersion typically represents about 85-95% w/w of the final composition.

The size of the nanoparticles is homogenized using conventional micro-fluidization techniques to further promote even dispersion in the Phase I solution. Following micro-fluidization, the size of the nanoparticles can range from about 50 nm to 200 nm in diameter.

A preferred embodiment of making the topical anesthetic composition adds a Phase I solution (about 10% w/w of the composition) with a Phase II dispersion (about 90% w/w of the composition) while mixing under moderate to vigorous agitation for a predetermined time period. The predetermined time period can be between 5 minutes to 60 minutes. In one embodiment, the predetermined time period is at least between 10 to 30 minutes. The resulting dispersion is pH adjusted to 4.5-6.5 with hydrochloric acid or sodium hydroxide as necessary and brought to the final weight of the composition with sterile water.

A preferred embodiment of the topical anesthetic composition comprises tetratcaine HCl 0.5%, lidocaine HCl 1.0%, benzalkonium chloride 0.005%, sterile water liposomes with cetylpyridinium chloride, lecithin, polysorbate 80, and a hydroxypropyl methycelulose base, and sterile water for injection. The pH of the resulting solution is adjusted to about 4.5-6.5. The final product is aseptically filtered through a 0.22 μm filter and packaged into pre-sterilized dispensers or containers.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods, and in the steps or in the sequence of steps of the methods described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

1. A topical anesthetic composition comprising:
a mixture of at least two anesthetic agents, wherein a first anesthetic agent is an ester-based anesthetic agent and a second anesthetic agent is an amide-based anesthetic agent;
a plurality of lipid-based positively charged nanoparticles; and
a viscous base.

2. The topical anesthetic composition according to claim 1, wherein the ester-based anesthetic agent is selected from the group consisting of procaine, amethocaine, cocaine, benzocaine, or tetracaine.

3. The topical anesthetic composition according to claim 2, wherein the ester-based anesthetic agent is tetracaine HCl.

4. The topical anesthetic composition according to claim 3, wherein the tetracaine HCl is present in concentrations ranging from about 0.01% w/w to 4.0% w/w.

5. The topical anesthetic composition according to claim 1, wherein the amide-based anesthetic agent is selected from the group consisting of lidocaine, mepivacaine, etidocaine, ropivacaine, or bupivacaine.

6. The topical anesthetic composition according to claim 4, wherein the amide-based anesthetic agent is lidocaine HCl.

7. The topical anesthetic composition according to claim 6, wherein the lidocaine HCl is present in concentrations ranging from about 0.05% w/w to 6.0% w/w.

8. The topical anesthetic composition according to claim 1, further comprising a preservative.

9. The topical anesthetic composition according to claim 8, wherein the preservative is benzalkonium chloride.

10. The topical anesthetic composition according to claim 1, wherein the viscous base comprises a cellulose base.

11. The topical anesthetic composition according to claim 10, wherein the cellulose base is selected from the group consisting of a hypromellose or hydroxypropyl methylcellulose, a microcrystalized cellulose, a methylcellulose, and mixtures thereof.

12. The topical anesthetic composition according to claim 10, wherein the viscous base further comprises a cation selected from the group consisting of cetylpyridinium chloride, cetyltrimethylammonium bromide, cetalkonium chloride, and mixtures thereof.

13. The topical anesthetic composition according to claim 10, wherein the viscous base further comprises one or more emulsifiers.

14. The topical anesthetic composition according to claim 13, wherein the emulsifier is lecithin.

15. The topical anesthetic composition according to claim 1, wherein the viscous base further comprises a nonionic surfactant.

16. The topical anesthetic composition according to claim 15, wherein the nonionic surfactant is polysorbate 80.

17. The topical anesthetic composition according to claim 1, wherein the plurality of positively charged nanoparticles are liposomes.

18. The topical anesthetic composition according to claim 17, wherein the liposome nanoparticles include a mixture of a lecithin, a polysorbate surfactant, and a cation selected from the group consisting of cetylpyridinium chloride, cetyltrimethylammonium bromide, cetalkonium chloride, and mixtures thereof.

19. The topical anesthetic composition according to claim 1, wherein the plurality of positively charged nanoparticles are nanoemulsions.

20. The topical anesthetic composition according to claim 19, wherein the nanoemulsions includes a mixture of mineral oil, a lecithin, a polysorbate surfactant, and a cation selected from the group consisting of cetylpyridinium chloride, cetyltrimethylammonium bromide, cetalkonium chloride, and mixtures thereof.

21. The topical anesthetic composition according to claim 1, further comprising sterile water.

22. The topical anesthetic composition according to claim 1, wherein the pH of the composition is about 4.5-6.5.
